(19) United States
(12) Patent Application Publication
(10) Pub. No.: US 2022/0273500 A1
(43) Pub. Date: Sep. 1, 2022

(54) FORMING SHRINKAGE FOLLOWING PORTION-BONDED MEDICAL TAPE AND METHOD FOR MANUFACTURING THE SAME

(71) Applicant: YUGENKAISHA CHOURYU, Shizuoka (JP)

(72) Inventor: Toshifumi HIRASHIMA, Shizuoka (JP)

(21) Appl. No.: 17/288,570

(22) PCT Filed: Mar. 12, 2021
(86) PCT No.: PCT/JP2021/010182

§ 371 Date: Apr. 26, 2021

(30) Foreign Application Priority Data
Oct. 30, 2020 (JP) 2020-182972

(51) Int. Cl. A61F 13/02

(52) U.S. Cl. CPC A61F 13/0283; A61F 13/0246; A61F 13/023

(57) ABSTRACT

A medical tape and the like having a high effect of alleviating a persistent skin irritation during an application duration caused by a residual shrink force associated with a forming shrinkage is provided. The medical tape includes an elastic base member portion that is film-formed of a solution polymer and has an elastic function, a forming shrinkage following portion having a following function followable to a forming shrinkage of the solution polymer, an adhesion portion having a function to apply and hold the elastic base member portion on a skin, and a release portion having a function to protect the adhesion portion. The forming shrinkage following portion, the elastic base member portion, the adhesion portion, and the release portion are laminated in this order.

### TECHNICAL FIELD

The present invention relates to a medical tape that deals with a forming shrinkage and a method for manufacturing the same.

### BACKGROUND ART

Conventionally, for medical industries or the like, a medical tape that uses a film material, such as a polyurethane film, having a function excellent in waterproof performance, moisture permeability, skin followability, and the like has been selected in order to cover and fix a gauze and a pad, fix a surgical tool, such as an indwelling needle or a catheter, cover and protect a wound or a skin, and the like. The indwelling needle is an injection needle used by being inserted into a vein and fixed to a body for blood sampling or drip infusion. The indwelling needle is also used in drip infusion or the like used for about a week.

Since these film materials have a very thin thickness of approximately 10 μm, it cannot independently hold a planar shape and is easily rounded under a natural environment. For this reason, as a support for holding the planar shape, a shape protection cover having stiffness higher than that of an elastic base member of the film material, such as an inelastic plastic film, is laminated with the film material in order to hold the planar shape and prevent formation of wrinkles or the like.

Note that, in its manufacturing process, as means to laminate the support for holding the planar shape and the film material, there has been widely used a method in which, after applying a mixed liquid (a urethane resin liquid and a crosslinking agent liquid) that serves as a raw material of the polyurethane film on an upper layer (one surface) of the inelastic plastic film, the mixed liquid is hardened to form a film, and thus, the polyurethane film is obtained, and simultaneously, the support for holding the planar shape and the film material are laminated.

However, since the above-described method simultaneously performs the film formation of the polyurethane film and the lamination on the support, there is generated a shrink force (an internal stress) remaining due to a forming shrinkage associated with the hardening of the mixed liquid serving as the raw material of the polyurethane film in the polyurethane film. Hereinafter, the “shrink force remaining in the elastic base member during the manufacturing process” will be referred to as a “residual shrink force.” Accordingly, in the medical tape that uses the polyurethane film obtained in the above-described method as the elastic base member of the medical tape and the inelastic plastic film as the support of the medical tape, that is, “the medical tape formed by simultaneously performing the film formation of the elastic base member in a film form and the lamination on the inelastic support positioned on an upper layer using the mixed liquid (the urethane resin liquid and the crosslinking agent liquid) serving as the raw material of the polyurethane film, applying an adhesion on a lower layer of the elastic base member, providing an inelastic release sheet on the lowermost layer, and providing an inelastic support for holding the planar shape on an upper layer of the elastic base member” (hereinafter, referred to as a “support-bonded medical tape using inelastic material as the support”), the residual shrink force associated with the forming shrinkage resides in the film material as the elastic base member. That is, when the mixed liquid serving as the raw material of the elastic base member is not attached on the support of the inelastic material, the mixed liquid serving as the raw material of the elastic base member freely shrinks, and thus, the residual shrink force is not generated. Meanwhile, when the mixed liquid serving as the raw material of the elastic base member is well attached on the support of the inelastic material, the mixed liquid serving as the raw material of the elastic base member cannot freely shrink except in a film thickness direction, and thus, the residual shrink force is generated near an attachment interface between the elastic base member and the support of the inelastic material after the film formation.

Note that the support-bonded medical tape using inelastic material as the support is applied in the following sequence: 1) remove the release sheet; 2) apply the tape on a skin; and 3) remove the support. In addition, the residual shrink force of the support-bonded medical tape using inelastic material as the support is not eliminated even by entirely removing the release sheet. After the tape is applied on the skin, the residual shrink force is released by removing the support, and acts as a force to continuously shrink the skin. Since the skin is shrunken, the residual shrink force causes a persistent skin irritation during the application duration.

Here, the residual shrink force existing in the support-bonded medical tape using inelastic material as the support will be described with reference to FIGS. 1 and 2. FIG. 1 is an explanatory diagram illustrating a state in which after a support-bonded medical tape using inelastic material as the support having a bisected support structure is applied on a skin, a support of the thumb side is removed. In order to compare states of the elastic base members underlying the support of the thumb side and the support of the fifth finger side, the support of the thumb side is removed, and the support of the fifth finger side is not removed. While fine wrinkles are observed in the elastic base member of the thumb side from which the support is removed after the tape is applied on the skin, no change is observed in the elastic base member of the fifth finger side from which the support is not removed. FIG. 2 illustrates a state in which the remaining support, that is, the support of the fifth finger side is removed. In FIG. 2, fine wrinkles are also observed in the elastic base member of the fifth finger side. It is conceived that this is because, as the support is removed from the support-bonded medical tape using inelastic material as the support, the elastic base member is released from the support and shrunken so as to exhibit fine wrinkles in the elastic base member, and therefore, a residual shrink force associated with the forming shrinkage exists in the elastic base member. In this manner, in the support-bonded medical tape using inelastic material as the support, the residual shrink force is released by removing the support. Therefore, the residual shrink force associated with the forming shrinkage existing in the support-bonded medical tape using inelastic material as the support can be observed by after applying the medical tape on the skin, removing the support to shrink the elastic base member, thereby causing fine wrinkles to appear in the elastic base member.

Note that, in the medical tape industry, a warning, such as “please apply without pulling (without stretching) because it may cause skin irritation,” is called in a handling manual or the like of the product. Therefore, in the medical tape industry, it is inferred that a risk of applying an elastic base member portion of an elastic medical tape on a skin in a “pulled and stretched state” should be sufficiently understood as a common sense of those skilled in the art. Additionally, in the plastic industry, it is conceived to be a common sense of those skilled in the art that applying and drying a solution polymer on an upper layer of an inelastic support (for example, a plastic film) and forming a polyurethane film and laminating it on the support at the same time generates the residual shrink force (the internal stress) associated with the forming shrinkage within the polyurethane film.

On the other hand, in the medical tape industry, it is inferred that there is little recognition (a common sense of those skilled in the art) that the residual shrink force associated with the forming shrinkage exits within the polyurethane film as the elastic base member portion of the support-bonded medical tape using inelastic material as the support. If there is the recognition (the common sense of those skilled in the art) of the existence of the residual shrink force associated with the forming shrinkage in the medical tape industry, and it is understood that the residual shrink force has a harmful effect, there should be a description of warning or the like on the “residual shrink force associated with the forming shrinkage existing in the elastic base member portion” in a handling manual or the like of the product. In a handling manual or the like of a medical tape currently manufactured and sold, there is only a warning that says “if a symptom that seems to be a skin disorder (rash, redness, itching, and the like) appears during use of this product, please stop using and carry out an appropriate treatment,” which is a warning that contains a problem of possibly misleading that it is caused by the user's constitutive endogenous cause. Accordingly, there has been a problem that, even though it is a medical tape used by a user with a purpose, the user himself uses the medical tape without recognizing the harmful effect associated with the “residual shrink force associated with the forming shrinkage existing in the elastic base member portion,” and moreover, without any preventive measures. Furthermore, a root of this problem is that there is a contradiction that the “residual shrink force associated with the forming shrinkage” is the common sense of those skilled in the art in the plastic industry but it is not the common sense of those skilled in the art in the medical tape industry where products using similar materials are manufactured and sold, and there has been a problem that no countermeasure has been taken for the residual shrink force associated with the forming shrinkage having the harmful effect in the medical tape industry.

In a part, a medical tape (for example, see Patent Document 1) for alleviating a persistent skin irritation during application duration of an elastic base member of an elastic medical tape has been proposed, and the persistent skin irritation during application duration of the support-bonded medical tape using inelastic material as the support caused by the residual shrink force is improved. However, this medical tape requires to laminate an elastic base member portion and an anti-expansion portion while the elastic base member portion is in a slacked state or in a not-pulled state in the manufacturing process, and therefore, the manufacturing of this medical tape raised a problem in manufacturing equipment, manufacturing cost, and the like from the aspect of efficiency. Accordingly, in the conventional support-bonded medical tape using inelastic material as the support, the production that deals with the residual shrink force associated with the forming shrinkage is not easy, but generates the residual shrink force associated with the forming shrinkage in the elastic base member portion of the medical tape. Thus, it is not easy to alleviate the persistent skin irritation during application duration caused by the residual shrink force associated with the forming shrinkage, and the current situation is that there has been no means that can appropriately solve such a disadvantage.

### CITATION LIST

### Patent Document

Patent Document 1: Japanese Patent No. 6312915

### SUMMARY OF INVENTION

In view of the aforementioned problems, it is therefore an object of the present invention to provide a medical tape that has a high effect of alleviating a persistent skin irritation during an application duration caused by a residual shrink force associated with a forming shrinkage of, in particular, a medical tape that has the forming shrinkage by using a solution polymer as a raw material and simultaneously performing a film formation and a lamination in a manufacturing process and aims to be applied on a skin that exhibits an excessive reaction even when a degree of residual shrink force associated with the forming shrinkage as a cause is an insignificant irritation so as to be described as of a level of magnifying lens or microscope, not as of a level of naked eye.

In order to address the aforementioned problems, the inventors made diligent efforts over and over and completed the present invention. That is, according to the present invention, there is provided a forming shrinkage following portion-bonded medical tape that includes an elastic base member portion, a forming shrinkage following portion, an adhesion portion, and a release portion. The elastic base member portion is film-formed of a solution polymer. The elastic base member portion has an elastic function. The forming shrinkage following portion has a following function followable to a forming shrinkage of the solution polymer. The adhesion portion has a function to apply and hold the elastic base member portion on a skin. The release portion has a function to protect the adhesion portion. The forming shrinkage following portion, the elastic base member portion, the adhesion portion, and the release portion are laminated in this order. Additionally, according to the present invention, there is provided a method for manufacturing a medical tape. The method includes a step of obtaining an elastic base member portion in a state where a residual shrink force generated in association with a forming shrinkage of a solution polymer is reduced by applying the solution polymer that turns into the elastic base member portion having an elastic function on an upper layer of a forming shrinkage following portion having a following function followable to the forming shrinkage of the solution polymer to simultaneously perform a film formation of the elastic base member portion and a lamination with the forming shrinkage following portion; and subsequently, a step of laminating an adhesion portion on the elastic base member portion and the adhesion portion has a function to apply and hold the elastic base member portion on a skin, and laminating a release portion on the adhesion portion and the release portion has a function to protect the adhesion portion. In the medical tape manufacturing method of the present invention, steps other than the above-described steps may be optional, and it is not specifically limited as long as the effect of the present invention can be provided.

### Advantageous Effects of Invention

The medical tape of the present invention ensures reducing the residual shrink force associated with the forming shrinkage in a manufacturing stage of the medical tape by introducing means for reducing the force to continuously shrink a skin caused by the residual shrink force associated with the forming shrinkage of, in particular, the medical tape that has the forming shrinkage by using the solution polymer as a raw material and simultaneously performing the film formation and the lamination in the manufacturing process to a medical tape as a function. Accordingly, when in use of the medical tape, the medical tape can be applied on a skin in a state where the residual shrink force associated with the forming shrinkage of the medical tape is reduced, and therefore, the force to continuously shrink the skin caused by the residual shrink force associated with the forming shrinkage of the medical tape can be reduced and the persistent skin irritation during the application duration caused by the residual shrink force can be alleviated. This ensures an application on the skin that exhibits an excessive reaction even when a degree of residual shrink force associated with the forming shrinkage as a cause is an insignificant irritation so as to be described as of a level of magnifying lens or microscope, not as of a level of naked eye, thereby ensuring providing a medical tape, an adhesive skin patch, and the like of low irritation. Furthermore, it is possible to prevent inflammation in sebaceous glands or sweat glands, inflammation in the root of hair or its surrounding tissues, a medical instrument pressure-related wound caused by poor blood circulation in skin cells, capillary vessels, or the like due to pressure, and the like. With the medical tape manufacturing method of the present invention, the medical tape of the present invention can be efficiently manufactured.

### BRIEF DESCRIPTION OF DRAWINGS

FIG. 1 is an explanatory diagram illustrating a state in which an inelastic support of a thumb side used for checking the residual shrink force described in paragraph [0007] is removed;

FIG. 2 is an explanatory diagram illustrating a state in which a support of a fifth finger side as a remaining inelastic support in the explanatory diagram illustrated in FIG. 1 is removed;

FIG. 3 is a drawing illustrating a manufacturing process of a medical tape according to a first embodiment of the present invention and is a schematic cross-sectional view illustrating a state of a lamination process in which a solution polymer 1 that later turns into an elastic base member portion 2 is applied to an upper layer of a forming shrinkage following portion 3;

FIG. 4 is a drawing illustrating the manufacturing process of the medical tape according to the first embodiment of the present invention and is a schematic cross-sectional view illustrating a state where a film formation of the elastic base member portion 2 and a lamination with the forming shrinkage following portion 3 and the elastic base member portion 2 are simultaneously performed by the solution polymer 1 applied on the upper layer of the forming shrinkage following portion 3 being hardened to terminate a forming shrinkage and a film of the solution polymer 1 being formed to be the elastic base member portion 2;

FIG. 5 is a schematic cross-sectional view illustrating the manufacturing process of the medical tape according to the first embodiment of the present invention, and illustrates a state where, after the forming shrinkage is terminated, an adhesion is applied on an upper layer of the elastic base member portion 2 to make an adhesion portion 4 and a release portion 5 is provided on an upper layer of the adhesion portion 4;

FIG. 6 is a schematic cross-sectional view illustrating the medical tape according to the first embodiment of the invention; and

FIG. 7 is a schematic cross-sectional view illustrating a state when in use of the medical tape according to the first embodiment of the invention, and illustrates a state where the release portion 5 is removed from the medical tape, the adhesion portion 4 is applied on a skin 6, and the forming shrinkage following portion 3 is removed.

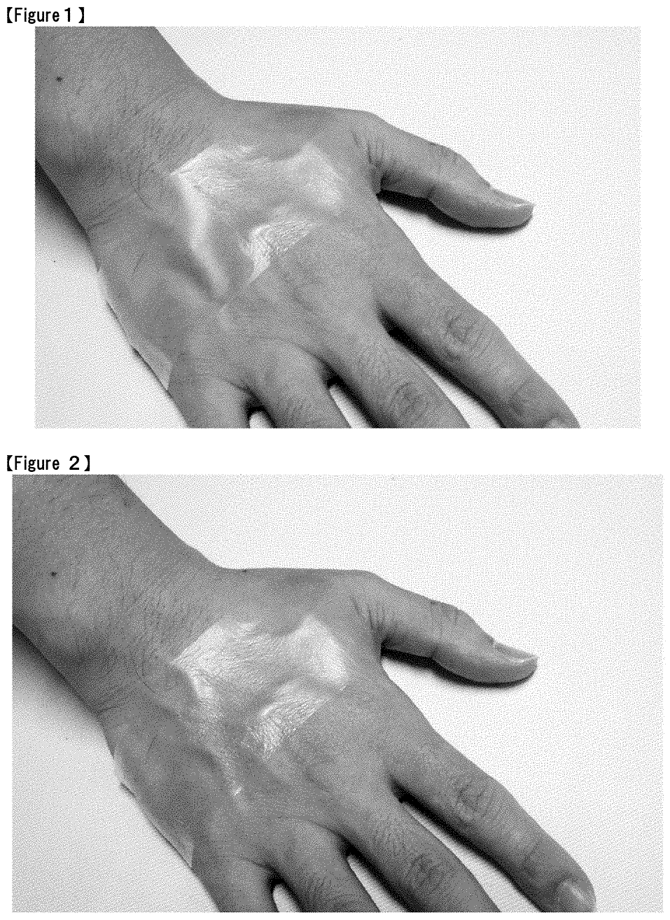

### DESCRIPTION OF EMBODIMENTS

In the course of diligent studies, the inventors found that a cause of a residual shrink force in a support-bonded medical tape using inelastic material as the support resided in a forming shrinkage caused by simultaneously performing a film formation and a lamination during manufacturing, and the residual shrink force of the support-bonded medical tape using inelastic material as the support was not eliminated even after a whole release sheet was removed, but after the medical tape was applied on a skin, the residual shrink force was released by removing the inelastic support to become a force that continuously shrinks the skin to shrink the skin, thereby causing a persistent skin irritation during application duration by the support-bonded medical tape using inelastic material as the support. Therefore, the inventors focused on, in the manufacturing process of the medical tape using a solution polymer as a raw material, means that reduces the residual shrink force associated with the forming shrinkage by dealing with the forming shrinkage caused by simultaneously performing the film formation and the lamination from a manufacturing stage.

Here, a skin irritation when the support-bonded medical tape using inelastic material as the support is used for the purpose to, for example, cover and protect wounds or skin will be described. The skin surface has openings of sebaceous glands or sweat glands and also hair. For this reason, the residual shrink force of the support-bonded medical tape using inelastic material as the support continuously deforms the openings of sebaceous glands or sweat glands and occasionally acts as an occluding force so as to generate inflammation (redness, swelling, fever, pain, and dysfunction) in sebaceous glands or sweat glands in some cases. In addition, the continuous skin shrinking force acts as a force to shrink a skin and persistently draw up the hair, so that inflammation occurs in the root of hair or its surrounding tissues in some cases.

Next, a skin irritation when the support-bonded medical tape using inelastic material as the support is used for the purpose to cover and fix a gauze, a pad and the like or fix a surgical tool, such as an indwelling needle or a catheter, will be described. The residual shrink force of the support-bonded medical tape using inelastic material as the support not only acts to fix the indwelling needle, the catheter, or the like but also acts as a force to persistently press a skin at the portion where the indwelling needle, the catheter, or the like is fixed. The residual shrink force is converted into a force of persistently pressing skin cells, capillary vessels, or the like via such surgical tools.

Next, the force of persistently pressing skin cells, capillary vessels, or the like will be described. For example, the blood vessels are stretched all over a human body, and its total extension length is approximately 100,000 km, which reaches around two and a half laps of the earth. In addition, nearly 95% of the blood vessels are capillary vessels. The capillary vessels have a diameter of approximately 7 μm and a wall thickness of 1 μm or smaller, which is significantly thin. For this reason, even for an unaware insignificant pressure, the capillary vessels of skin may be easily deformed or obstructed.

There are approximately five million red corpuscles, used to carry oxygen to cells over the entire body, per a microliter of blood. The red corpuscles are disk-shaped solid materials having recessed centers on both sides with a diameter of approximately 7 to 8 μm and a thickness of approximately 2 μm. The red corpuscles pass through the capillary vessels having a diameter of approximately 7 μm while they are deformed. However, since deformation of the red corpuscles as solid materials is limited, it is difficult to allow the red corpuscles to pass through the capillary vessels even when slight deformation occurs in capillary vessels. In addition, the capillary vessels may be clogged due to occlusion of red corpuscles in some cases. As a result, oxygen deficiency often occurs in cells due to poor blood circulation.

Poor blood circulation caused by pressure may generate a disease in which tissues or cells of skin locally die, such as a decubitus, so called a bedsore. Experimentally, it is said that a decubitus is generated if a persistent pressure is applied to a same part of a body for two hours or longer. For this reason, in order to prevent the decubitus from occurring in the medical industry, it is recommended that a posture be changed at approximately two hour intervals for a bedridden patient who is in a state of lying on a bed or the like, and at approximately thirty minute intervals for a patient who in a state of sitting on a wheelchair or the like. In this manner, the decubitus is generated due to poor blood circulation caused by pressure for several hours.

Furthermore, the recent support-bonded medical tapes using inelastic material as the support have been remarkably improved in skin followability, moisture permeability, waterproof property, skin adherence, and the like, compared with the conventional elastic medical tapes, so that they can be continuously bonded for about a week. For this reason, the support-bonded medical tape using inelastic material as the support are used in many cases to cover and fix a gauze, a pad, and the like, and in particular, fix an indwelling needle for drip infusion or the like for about a week. The force of persistently shrinking skin by covering the surgical tool acts as a force to persistently press a portion of the skin where a medical instrument, such as the indwelling needle or the catheter, is fixed for several days. Therefore, a medical instrument pressure-related wound and the like may occur in the fixed portion. This is inferred to be a period of time far exceeding a limitation of several hours for preventing the decubitus from occurring.

Due to improved qualities of adhesives and elastic base members, a long-time fixation with peel-off durability has been possible, and there has been a product that can be attached for a week or longer. In such a product less likely to be peeled off and excellent for long-time fixation, it is important to inform a warning “when removing the product, please gently remove the product along hair streaks so as not to harm skin” in the handling manual or the like. In order to alleviate skin irritation at the time of removing the product, means that does not forcibly peel the product but leaves it until it peels off is effective. However, use of the means that leaves the product until it peels off naturally increases the application duration, and the persistent skin irritation during the application duration caused by the residual shrink force continuously increases.

Note that, even for insignificant irritation, many patients complain of discomfort and distress. However, if insignificant irritation is persistently applied to the body, “hypaesthesia” which causes dull sensation to a stimulus occurs, so that the number of patients who complain of discomfort or distress is reduced. Although this is generally called “habituation,” it does not mean that the stimulus to the body has disappeared, and deformation or occlusion generated in capillary vessels of skin, poor blood circulation, and the like are improved. Even though a degree of the residual shrink force associated with the forming shrinkage described in the paragraphs [0017] to [0023] is an insignificant irritation that may be described as of a level of magnifying lens or microscope, not as of a level of naked eye, it can sufficiently be such a cause.

In this manner, in order to alleviate persistent skin irritation during the application duration, it is important to take the process that leads to the forming shrinkage into consideration and reduce the force of persistently shrinking skin caused by the residual shrink force associated with the forming shrinkage such that the residual shrink force associated with the forming shrinkage is not generated during the film formation in the elastic base member portion of the forming shrinkage following portion-bonded medical tape. Here, in the manufacturing process of the forming shrinkage following portion-bonded medical tape, the film formation and the lamination with the forming shrinkage following portion are simultaneously performed by applying the solution polymer that later turns into the elastic base member portion on the upper layer of the forming shrinkage following portion. At this time, the forming shrinkage associated with the hardening of the solution polymer occurs on the upper layer of the forming shrinkage following portion, the solution polymer is hardened, and the forming shrinkage is terminated, then, the solution polymer film is formed to be the elastic base member portion. In this process, a length of the elastic base member portion is gradually shortened, an upper base of the forming shrinkage following portion that shrinks the elastic base member portion on surface follows the length of the elastic base member portion and shrinks. On the other hand, a lower base of the forming shrinkage following portion that does not contact the elastic base member portion on surface is less likely to be affected by the forming shrinkage of the elastic base member portion as much as the upper base of the forming shrinkage following portion is affected, and therefore, it does not follow the shrinkage of the elastic base member portion, and thus, does not shrink. Accordingly, the forming shrinkage following portion has a cross-sectional surface in a trapezoidal shape with the upper base being shorter than the lower base. This process ensures obtaining the elastic base member portion in a state where the residual shrink force caused in association with the forming shrinkage of the solution polymer is reduced or eliminated. Providing the elastic base member portion obtained by the above-described method with the adhesion portion and the release portion completes the forming shrinkage following portion-bonded medical tape in a state where the residual shrink force caused in association with the forming shrinkage of the solution polymer is reduced or eliminated.

The forming shrinkage following portion-bonded medical tape manufactured as described above is used in the following sequence: 1) remove the release portion; 2) apply the adhesion portion on a skin; and 3) remove the forming shrinkage following portion. Thus, dealing with the forming shrinkage from the manufacturing stage ensures using the forming shrinkage following portion-bonded medical tape in the state where the residual shrink force caused in association with the forming shrinkage of the solution polymer is reduced or eliminated. Accordingly, in manufacturing the forming shrinkage following portion-bonded medical tape, reducing the residual shrink force reduces a force to continuously shrinks a skin caused by the residual shrink force of the conventional support-bonded medical tape using inelastic material as the support, in particular, the medical tape that has the forming shrinkage by using the solution polymer as a raw material and simultaneously performing the film formation and the lamination in the manufacturing process, thereby providing the effective means for alleviating the persistent skin irritation during the application duration.

Introducing these effective means to the forming shrinkage following portion-bonded medical tape as a function ensures applying the medical tape on a skin with the residual shrink force of the medical tape being reduced when in use of the medical tape, reducing the force that continuously shrinks the skin due to the residual shrink force of the medical tape, and thus, alleviating the persistent skin irritation during the application duration caused by the residual shrink force, thereby ensuring an application on a skin that exhibits an excessive reaction even with an insignificant irritation. Furthermore, it can be expected that a skin trouble, a medical instrument pressure-related wound, or the like caused by the persistent skin irritation during the application duration is prevented. From this viewpoint, it would be recognized that the conventional support-bonded medical tape using inelastic material as the support, in particular, the medical tape that has the forming shrinkage by using the solution polymer as the raw material and simultaneously performing the film formation and the lamination in the manufacturing process fail to consider the residual shrink force associated with the forming shrinkage.

Therefore, in order to provide the forming shrinkage following portion-bonded medical tape that takes in a function to reduce the residual shrink force associated with the forming shrinkage of the conventional support-bonded medical tape using inelastic material as the support, in particular, the medical tape that has the forming shrinkage by using the solution polymer as the raw material and simultaneously performing the film formation and the lamination in the manufacturing process, the inventors selected a material that could be laminated with the forming shrinkage following portion from materials that hardened and formed a film after being applied on the upper layer of the forming shrinkage following portion as a material of the solution polymer later turning into the elastic base member portion, and furthermore, selected a material usable for medical treatment and excellent in waterproof performance, moisture permeability, skin followability, and the like from materials that functioned as the elastic base member portion after the film formation. Next, as the material of the forming shrinkage following portion, the inventors selected a material that had a following function followable to the forming shrinkage of the solution polymer. As the material of the adhesion portion, the inventors selected a medical adhesive that had a function to apply and hold the elastic base member portion on a skin and caused little adhesive residue, keratin damage or the like at the time of removal, and furthermore, as the material of the release portion, the inventors selected a material that had a function to protect the adhesion portion. Thus, the inventors conceived to configure the forming shrinkage following portion-bonded medical tape using the functions included in the respective materials.

By combining these materials, the film formation of the elastic base member portion and the lamination with the forming shrinkage following portion are simultaneously performed by applying the solution polymer that later turns into the elastic base member portion on the upper layer of the forming shrinkage following portion in the manufacturing process of the forming shrinkage following portion-bonded medical tape. Then, the forming shrinkage associated with the hardening of the solution polymer gradually shortens the length of the elastic base member portion, and the upper base (a surface in contact with the elastic base member portion) of the forming shrinkage following portion shrinks by following the length of the elastic base member portion. As a result, a cross-sectional surface of the forming shrinkage following portion has a trapezoidal shape with the upper base shorter than the lower base (a surface not in contact with the elastic base member portion). This ensures obtaining the elastic base member portion in the state where the residual shrink force caused in association with the forming shrinkage of the solution polymer is reduced or eliminated. Then, providing the adhesion portion and the release portion to the elastic base member portion obtained by the above-described method ensures manufacturing the forming shrinkage following portion-bonded medical tape in the state where the residual shrink force caused in association with the forming shrinkage of the solution polymer is reduced or eliminated. Accordingly, the fact that it is possible to apply the medical tape on a skin in a state where the residual shrink force is reduced or eliminated when in use of the forming shrinkage following portion-bonded medical tape, to introduce the means for reducing the force to continuously shrink the skin caused by the residual shrink force of the conventional support-bonded medical tape using inelastic material as the support, in particular, the medical tape that has the forming shrinkage by using the solution polymer as the raw material and simultaneously performing the film formation and the lamination in the manufacturing process to the forming shrinkage following portion-bonded medical tape as a function, and to provide the medical tape that can be applied on the skin that exhibits an excessive reaction even with an insignificant irritation by alleviating the persistent skin irritation during the application duration caused by the residual shrink force. Thus, the present invention reached the completion.

The medical tape of the present invention includes the forming shrinkage following portion, the elastic base member portion, the adhesion portion, and the release portion in this order from the upper layer. In the manufacturing process, the film formation of the elastic base member portion and the lamination with the forming shrinkage following portion are simultaneously performed by applying the solution polymer that later turns into the material of the elastic base member portion on the upper layer of the forming shrinkage following portion. Then, the forming shrinkage associated with the hardening of the solution polymer gradually shortens the length of the elastic base member portion, and the upper base (the surface in contact with the elastic base member portion) of the forming shrinkage following portion shrinks by following the length of the elastic base member portion. As a result, the cross-sectional surface of the forming shrinkage following portion has the trapezoidal shape with the upper base shorter than the lower base (the surface not in contact with the elastic base member portion) as illustrated in FIG. 4. This ensures obtaining the elastic base member portion in the state where the residual shrink force caused in association with the forming shrinkage of the solution polymer is reduced or eliminated, and providing the adhesion portion and the release portion on the upper layer of the elastic base member portion completes the forming shrinkage following portion-bonded medical tape in the state where the residual shrink force caused in association with the forming shrinkage of the solution polymer is reduced or eliminated, thereby ensuring using the forming shrinkage following portion-bonded medical tape in the state where the residual shrink force is reduced or eliminated.

Note that, a timing to provide the adhesion portion and the release portion on the upper layer of the elastic base member portion is preferred to be after the forming shrinkage is terminated (after the film formation is terminated) when the residual shrink force associated with the forming shrinkage of the elastic base member portion has been eliminated. However, it is not necessary to wait until the film formation is terminated by taking materials of the forming shrinkage following portion and the solution polymer to be selected, manufacturing equipment, production efficiencies, and the like into consideration. Even if the residual shrink force associated with the forming shrinkage of the solution polymer remained in a boundary portion between the elastic base member portion and the adhesion portion as the result of not waiting until the film formation is terminated and providing the inelastic release portion having the adhesion portion immediately after the application of the solution polymer on the forming shrinkage following portion, the above-described residual shrink force associated with the forming shrinkage is eliminated by removing the release portion when in use. Therefore, it can be used in the state where the residual shrink force is reduced or eliminated when in use. Accordingly, the timing to provide the adhesion portion and the release portion may be any timing.

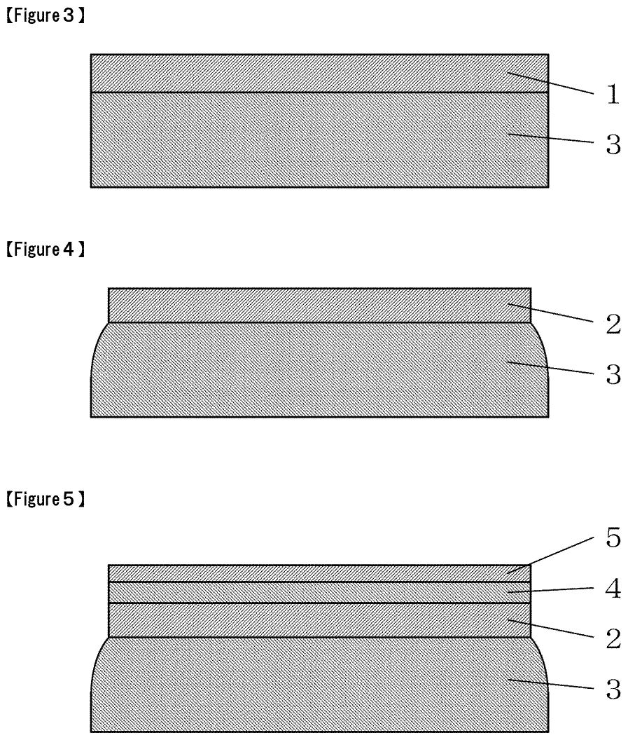

In the medical tape of the present invention, the solution polymer that later turns into the elastic base member portion is applied on the upper layer of the forming shrinkage following portion. At this time, the cross-sectional shapes of the forming shrinkage following portion and the solution polymer are in rectangular shapes having the same lengths (FIG. 3). Then, the forming shrinkage following portion positioned on the lower layer follows the shrinkage of the solution polymer and gradually shrinks in association with the forming shrinkage of the solution polymer, and at the point when the forming shrinkage of the solution polymer is terminated, the shrinkage of the forming shrinkage following portion is also terminated (FIG. 4). This ensures obtaining the elastic base member portion in the state where the residual shrink force caused in association with the forming shrinkage of the solution polymer is reduced or eliminated. Then, manufacturing the forming shrinkage following portion medical tape using the elastic base member portion obtained in the above-described process ensures reducing or eliminating the residual shrink force residing in the forming shrinkage following portion medical tape. Note that, a degree of reduced residual shrink force is preferred to be a complete elimination. However, a degree of residual shrink force associated with the forming shrinkage as a cause is as insignificant as to be described as of a level of magnifying lens or microscope, not as of a level of naked eye, and thus, the residual shrink force to be reduced may be insignificant and the effect of the invention can be sufficiently provided even if it is insignificant.

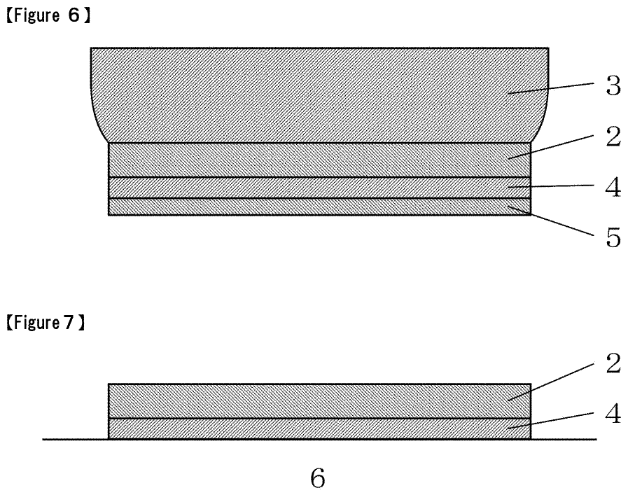

While examples of the invention will now be described in details with reference to the accompanying drawings, the invention is not limited thereto. According to the present invention, in appropriately selecting and combining the materials that constitute the forming shrinkage following portion-bonded medical tape and in the manufacturing process, a soft polyurethane foam in a film shape having a following function followable to the forming shrinkage of the solution polymer is selected as the forming shrinkage following portion, and the solution polymer (a urethane resin liquid and a crosslinking agent liquid) as a raw material of the polyurethane film that later turns into the elastic base member portion is applied on the upper layer of the forming shrinkage following portion (FIG. 3), after the solution polymer is hardened to be the elastic base member portion, the forming shrinkage is terminated, and the residual shrink force in the elastic base member portion is eliminated (FIG. 4), an acrylic adhesive is applied as the adhesion portion having a function to apply and hold the elastic base member portion on a skin and the release portion formed by coating a release agent on a surface of a high-quality paper is provided (FIG. 5), and thus, the forming shrinkage following portion-bonded medical tape is completed (FIG. 6). Note that, in the medical tape, the forming shrinkage following portion is removed after the adhesion portion is applied on the skin when in used (FIG. 7). The forming shrinkage following portion-bonded medical tape is based on reducing the force to continuously shrinks a skin caused by the residual shrink force and alleviating the persistent skin irritation during the application duration caused by the residual shrink force. The present invention needs to reduce the residual shrink force when the forming shrinkage following portion medical tape is manufactured.

A forming shrinkage following portion-bonded medical tape of a first embodiment of the present invention illustrated in FIG. 3 to FIG. 7 is configured of a solution polymer (a urethane resin liquid and a crosslinking agent liquid) 1, an elastic base member portion 2, a forming shrinkage following portion 3, an adhesion portion 4, and a release portion 5. The solution polymer 1 is a raw material of the elastic base member portion 2. The elastic base member portion 2 is film-formed by the solution polymer (the urethane resin liquid and the crosslinking agent liquid) 1 as the raw material of the elastic base member portion 2 and has the elastic function. The forming shrinkage following portion 3 has the following function followable to the forming shrinkage of the solution polymer 1. The adhesion portion 4 has the function to apply and hold the elastic base member portion 2 on a skin. The release portion 5 has the function to protect the adhesion portion 4.

FIG. 3 is a drawing that illustrates a manufacturing process of the forming shrinkage following portion-bonded medical tape according to the first embodiment of the present invention, and is a schematic cross-sectional view illustrating a state of lamination process in which the solution polymer as the material of the elastic base member portion is applied on the upper layer of the forming shrinkage following portion. In the forming shrinkage following portion-bonded medical tape as the first embodiment, the film formation of the elastic base member portion 2 and the lamination with the forming shrinkage following portion 3 are simultaneously performed by selecting the soft polyurethane foam in a film form having the following function followable to the forming shrinkage of the solution polymer 1 as the forming shrinkage following portion 3 and applying the solution polymer (the urethane resin liquid and the crosslinking agent liquid) 1 as the raw material of the polyurethane film that later turns into the elastic base member portion 2 on the upper layer of the forming shrinkage following portion 3. At this time, lengths of the forming shrinkage following portion 3 and the solution polymer 1 are in the same lengths as illustrated in FIG. 3. Note that, the reference numeral 1 denotes the solution polymer and the reference numeral 3 denotes the forming shrinkage following portion.

FIG. 4 is a schematic cross-sectional view illustrating the manufacturing process of the medical tape according to the first embodiment of the present invention, and illustrating a state of the forming shrinkage following portion 3 and the elastic base member portion 2 at a time point where the solution polymer 1 is hardened to complete the film formation and the forming shrinkage is terminated from the state where the solution polymer 1 that later turns into the elastic base member portion 2 is applied on the upper layer of the forming shrinkage following portion 3 illustrated in FIG. 3. At the time point illustrated in FIG. 3, the forming shrinkage following portion 3 and the solution polymer 1 had the same lengths. Thereafter, as illustrated in FIG. 4, the length of the elastic base member portion 2 was gradually shortened by the forming shrinkage associated with the hardening of the solution polymer 1, and the forming shrinkage was also terminated at the time point where the film formation was completed. The forming shrinkage following portion 3 had an upper base (a surface in contact with the elastic base member portion 2) whose length was shortened by following the shrinkage of the elastic base member portion 2, and the forming shrinkage following portion 3 had a cross-sectional surface in a trapezoidal shape with the upper base (the surface in contact with the elastic base member portion 2) being shorter than a lower base (a surface not in contact with the elastic base member portion 2). This indicates that the generation of the residual shrink force that was difficult to prevent from occurring in prior art in which the film formation of the elastic base member portion 2 and the lamination to an inelastic support were simultaneously performed and the residual shrink force of an amount corresponding to a difference between the lower base and the upper base of the forming shrinkage following portion 3 could be prevented from occurring by the forming shrinkage following portion-bonded medical tape of the invention. Furthermore, it indicates that the residual shrink force associated with the forming shrinkage could be reduced compared with the prior art. Note that the reference numeral 2 denotes the elastic base member portion and the reference numeral 3 denotes the forming shrinkage following portion.

FIG. 5 is a schematic cross-sectional view illustrating the manufacturing process of the medical tape according to the first embodiment of the present invention. On the upper layer of the elastic base member portion 2 in which the forming shrinkage has been terminated illustrated in FIG. 4, an adhesion is applied as the adhesion portion 4 having the function to apply and hold the elastic base member portion 2 on a skin, and the release portion 5 having the function to protect the adhesion portion 4 is provided on the upper layer of the adhesion portion 4, and thus, the forming shrinkage following portion medical tape is completed. Note that the reference numeral 2 denotes the elastic base member portion, the reference numeral 3 denotes the forming shrinkage following portion, the reference numeral 4 denotes the adhesion portion, and the reference numeral 5 denotes the release portion.

FIG. 6 is a schematic cross-sectional view of the medical tape according to the first embodiment of the present invention, and FIG. 7 is a schematic cross-sectional view illustrating a state when in use of the medical tape. FIG. 7 illustrates a state where the release portion 5 is removed, the adhesion portion 4 is applied on a skin 6, and the forming shrinkage following portion 3 is removed from the state illustrated in FIG. 6. Note that the reference numeral 2 denotes the elastic base member portion, the reference numeral 3 denotes the forming shrinkage following portion, the reference numeral 4 denotes the adhesion portion, the reference numeral 5 denotes the release portion, and the reference numeral 6 denotes the skin.

The forming shrinkage following portion-bonded medical tape is used in the following sequence: 1) remove the release portion; 2) apply the adhesion portion on a skin; and 3) remove the forming shrinkage following portion. In the forming shrinkage following portion-bonded medical tape ensures obtaining the elastic base member portion from which the residual shrink force associated with the forming shrinkage is reduced or eliminated compared with the prior art by dealing with the generation of the residual shrink force associated with the forming shrinkage of the elastic base member portion that has been difficult to prevent from occurring in the prior art in the manufacturing process and preventing the generation of the residual shrink force of the amount corresponding to the difference between the lower base and the upper base of the forming shrinkage following portion laminated on the elastic base member portion. Therefore, a clear change such as a shrinkage of the elastic base member portion does not occur as it occurs in the support-bonded medical tape using inelastic material as the support in the prior art even though the forming shrinkage following portion is removed after applied on a skin.

In the forming shrinkage following portion-bonded medical tape, the residual shrink force of the amount corresponding to the difference between the lower base and the upper base of the forming shrinkage following portion laminated on the elastic base member portion is prevented from being generated in the manufacturing process, and the elastic base member portion in the state where the residual shrink force associated with the forming shrinkage is reduced or eliminated compared with the prior art is obtained, and thus, the forming shrinkage following portion-bonded medical tape is completed. Therefore, the forming shrinkage following portion-bonded medical tape can be applied on a skin in the state where the residual shrink force is reduced. Accordingly, the force that continuously shrinks a skin due to the residual shrink force of the forming shrinkage following portion-bonded medical tape is reduced, thereby ensuring alleviating the persistent skin irritation during the application duration. Accordingly, in the forming shrinkage following portion-bonded medical tape of the present invention, by introducing the means for reducing the force to continuously shrinks a skin caused by the residual shrink force associated with the forming shrinkage of the support-bonded medical tape using inelastic material as the support in the prior art to a medical tape as a function, it is possible to apply the medical tape on a skin in the state where the residual shrink force of the forming shrinkage following portion-bonded medical tape is reduced when in use of the forming shrinkage following portion-bonded medical tape, reduce the force to continuously shrinks a skin due to the residual shrink force of the forming shrinkage following portion-bonded medical tape, and alleviate the persistent skin irritation during the application duration caused by the residual shrink force. This ensures an application on the skin that exhibits an excessive reaction even when a degree of residual shrink force associated with the forming shrinkage as a cause is an insignificant irritation so as to be described as of a level of magnifying lens or microscope, not as of a level of naked eye, thereby ensuring providing a medical tape, an adhesive skin patch, and the like of low irritation.

Specifically, in the first embodiment of the present invention, the urethane resin liquid and the crosslinking agent liquid are the solution polymer 1, the polyurethane film that is film-formed of the solution polymer 1 and has the elastic function is the elastic base member portion 2, the soft polyurethane foam in a film form having the following function followable to the forming shrinkage of the solution polymer 1 is the forming shrinkage following portion 3, the acrylic adhesive having the function to apply and hold the elastic base member portion 2 on a skin is the adhesion portion 4, and the release sheet formed by coating the release agent on the surface of the high-quality paper is the release portion 5. In the manufacturing process, the film formation of the polyurethane film that turns into the elastic base member portion 2 and the lamination with the soft polyurethane foam in the film form are simultaneously performed by applying the urethane resin liquid and the crosslinking agent liquid as the solution polymer 1 on the upper layer of the soft polyurethane foam in the film form as the forming shrinkage following portion 3, and then, the length of the polyurethane film is gradually shortened by the forming shrinkage associated with the hardening of the urethane resin liquid and the crosslinking agent liquid, and the upper base (the surface in contact with the polyurethane film) of the soft polyurethane foam in the film form shrinks by following the length of the polyurethane film. As a result, the cross-sectional surface of the soft polyurethane foam in the film form has the trapezoidal shape with the upper base being shorter than the lower base (the surface not in contact with the polyurethane film), and after the film formation of the polyurethane film and the lamination with the soft polyurethane foam in the film form are terminated, the acrylic adhesive is provided on the upper layer of the polyurethane film and the release sheet formed by coating the release agent on the surface of the high-quality paper is provided on the upper layer of the adhesion. Note that, while it is not illustrated, as a pretreatment of the example of the present invention, the solution polymer as the raw material of the polyurethane film that later turns into the elastic base member portion is ultra-thinly applied so as not to inhibit the following function of the forming shrinkage following portion on the upper layer of the soft polyurethane foam in the film form selected as the forming shrinkage following portion in order to include roles of preventing the solution polymer from penetrating into the soft polyurethane foam in the film form and of an adhesion for laminating both of them, and the solution polymer is hardened to form the film.

While in the first embodiment, the urethane resin liquid and the crosslinking agent liquid are used as the material of the solution polymer, any solution polymer material may be used as long as it can be applied on the upper layer of the forming shrinkage following portion and laminated with the material of the forming shrinkage following portion and can have the elastic function after the film formation and be provided with the adhesion portion on the upper layer. The solution polymer material may be any solution polymer materials that can provide functions appropriate for the respective usages, such as covering and fixing a gauze, a pad, and the like, fixing a surgical tool, such as an indwelling needle, a catheter, or the like, and covering and protecting wounds or skin, another new material and the like may be used, or different solution polymer materials may be mixed or laminated. The soft polyurethane foam in the film form having the following function followable to the forming shrinkage of the solution polymer is used as the material of the forming shrinkage following portion, a material having a nonwoven-fabric structure, a raised-fiber structure, or the like may be used as long as the material has the following function followable to the forming shrinkage of the solution polymer. It is not limited to a material that has the following function followable to the forming shrinkage of the solution polymer by itself alone, such as the soft polyurethane foam in the film form, the nonwoven-fabric structure, and the raised-fiber structure. For example, a material having a expansion and contraction function, such as a plate-shaped rubber, may be applied with an external force and pulled and stretched equally to a forming shrinkage rate of the solution polymer, the solution polymer may be applied on its upper layer, the applied external force may be removed after the forming shrinkage of the solution polymer is terminated (after the film formation is terminated) to follow the forming shrinkage of the solution polymer, and the residual shrink force associated with the forming shrinkage may be reduced or eliminated, or the residual shrink force associated with the forming shrinkage may be reduced or eliminated by the combination of other materials. The material is not specifically limited as long as the effect of the present invention can be provided. While the acrylic adhesive is used for the material of the adhesion portion, the material of the adhesion portion may be any adhesion that has the function to apply and hold the elastic base member portion on a skin and is usable for medical treatment, and the adhesion may be applied partly or entirely to correspond to the usage, and thus, the material, the shape, and the like are not restricted. While the release sheet formed by coating the release agent on the surface of the high-quality paper is used for the material of the release portion, the material, the shape, and the like are not restricted to correspond to the usage, design, convenience, and the like as long as the material of the release portion has the function to protect the adhesion portion.

Note that a support may be provided on the upper layer of the forming shrinkage following portion of the forming shrinkage following portion-bonded medical tape to correspond to the usage. For example, the support may be provided for the purpose of holding the planar shape of the medical tape so as not to form wrinkles and the like or an inelastic plastic film or the like may be provided for, for example, the purpose to prevent a returning force (a recoiling shrink force) of the elastic base member from acting caused by using the pulled and stretched elastic base member when in use of the medical tape. Note that the support may have any material, shape, and the like to correspond to the usage, design, convenience, and the like and is not specifically limited as long as the effect of the present invention can be provided.

The medical tape of the present invention illustrated in FIG. 3 to FIG. 7 all has the basic structure of the present invention. Such a configuration ensures manufacturing the medical tape from which the residual shrink force associated with the forming shrinkage of the solution polymer is reduced in the manufacturing process by introducing the means for reducing the force to continuously shrink a skin caused by the residual shrink force associated with the forming shrinkage of the support-bonded medical tape using inelastic material as the support in the prior art to the medical tape as a function. Therefore, the forming shrinkage following portion-bonded medical tape can alleviate the persistent skin irritation during the application duration caused by the residual shrink force by applying the medical tape on a skin in the state where the residual shrink force associated with the forming shrinkage is reduced and reducing the force to continuously shrink a skin caused by the residual shrink force associated with the forming shrinkage of the forming shrinkage following portion-bonded medical tape. This ensures an application on the skin that exhibits an excessive reaction even when a degree of residual shrink force associated with the forming shrinkage as a cause is an insignificant irritation so as to be described as of a level of magnifying lens or microscope, not as of a level of naked eye, thereby ensuring providing a medical tape, an adhesive skin patch, and the like of low irritation. Preventions of inflammation in sebaceous glands or sweat glands, inflammation in hair roots and surrounding tissues, medical instrument pressure-related wounds caused by poor blood circulation of cells of skin, capillary blood vessels, and the like caused by pressure, and the like can be expected.

While the example where the medical tape has a shape of rectangular has been described in the first embodiment above, any shape can be selected for the shape of the medical tape to correspond to the usage. For example, a rolled shape and a sheet shape are possible, and a shape that fits each part of body is also possible. While the medical tape is basically configured of the forming shrinkage following portion, the elastic base member portion, the adhesion portion, and the release portion from the upper layer, a gauze, a pad, and the like may be provided on the lower layer of the adhesion portion to correspond to the usage, design, convenience, and the like. Note that while the present invention is based on the application on a skin in a closely contacted manner, the medical tape may be applied via a protecting material and the like for protecting the skin from the medical adhesive and the like in some cases.

While all the residual shrink force associated with the forming shrinkage is preferred to be eliminated, a state where it is slightly reduced is also possible. This is because, as described in the paragraphs [0017] to [0024], the degree of residual shrink force associated with the forming shrinkage that this patent is solving is not of a level of naked eye, but is of a level of magnifying lens or microscope. In this description, the process from the manufacturing of the medical tape to the application is described, and “when in use” and “during application duration” are defined as follows. “When in use” represents the duration from removing the release portion, applying on a skin, until removing the forming shrinkage following portion following the procedure of using the medical tape. “During application duration” represents the duration where the forming shrinkage following portion-bonded medical tape is continuously applied.

Note that the function of the medical tape according to the present invention is effectively applied to a medical tape aiming to be applied to a human body. For example, since a skin irritation hypersensitivity patient severely reacts to even an insignificant skin irritation, the use of medical tapes containing percutaneously absorbable drugs is postponed in some cases. However, since it is possible to alleviate the insignificant skin irritation by using the forming shrinkage following portion-bonded medical tape according to the present invention, the medical tape according to the present invention can be used as a patch by blending percutaneously absorbable drugs in the adhesive portion. Providing a pad to the medical tape of the present invention ensures a usage as a treatment product.

### INDUSTRIAL APPLICABILITY

The forming shrinkage following portion-bonded medical tape according to the present invention has an extremely high effect of alleviating the persistent skin irritation during the application duration caused by the residual shrink force associated with the forming shrinkage of the support-bonded medical tape using inelastic material as the support in the prior art, in particular, the medical tape that has the forming shrinkage by using the solution polymer as the raw material and simultaneously performing the film formation and the lamination in the manufacturing process so as to be applicable on the skin that exhibits an excessive reaction even with an insignificant irritation. In addition, preventions of a skin trouble, a medical instrument pressure-related wound, and the like caused by the persistent skin irritation during the application duration can be expected. Furthermore, it is possible to provide a low-irritation medical tape, patch or the like. Therefore, the medical tape according to the present invention will greatly contribute to the medical industries.

### REFERENCE SIGNS LIST

1 solution polymer
2 elastic base member portion
3 forming shrinkage following portion
4 adhesion portion
5 release portion
6 skin

1. A medical tape comprising:
an elastic base member portion film-formed of a solution polymer, the elastic base member portion having an elastic function;
a forming shrinkage following portion having a following function followable to a forming shrinkage of the solution polymer;
an adhesion portion having a function to apply and hold the elastic base member portion on a skin; and
a release portion having a function to protect the adhesion portion,
wherein the forming shrinkage following portion, the elastic base member portion, the adhesion portion, and the release portion are laminated in this order.

2. A method for manufacturing a medical tape comprising:
a step of obtaining an elastic base member portion in a state where a residual shrink force generated in association with a forming shrinkage of a solution polymer is reduced by applying the solution polymer that turns into the elastic base member portion having an elastic function on an upper layer of a forming shrinkage following portion having a following function followable to the forming shrinkage of the solution polymer to simultaneously perform a film formation of the elastic base member portion and a lamination with the forming shrinkage following portion; and
subsequently, a step of laminating an adhesion portion on the elastic base member portion, the adhesion portion having a function to apply and hold the elastic base member portion on a skin, and laminating a release portion on the adhesion portion, the release portion having a function to protect the adhesion portion.
